(19) United States
(12) Patent Application Publication
(10) Pub. No.: US 2021/0290862 A1
(43) Pub. Date: Sep. 23, 2021

(54) DIGITAL DOSAGE OF AQUEOUS MEDICINAL SOLUTIONS

(71) Applicant: Ronin Labs LLC, Woodbridge, VA (US)

(72) Inventors: Michael Darling, Woodbridge, VA (US); Andrei Tapliga, Wall, NJ (US); Chuhao Zhou, Everett, MA (US)

(21) Appl. No.: 17/203,818

(22) Filed: Mar. 17, 2021

Related U.S. Application Data
(60) Provisional application No. 62/991,119, filed on Mar. 18, 2020.

(51) Int. Cl. A61M 11/00; G16H 20/13

(52) U.S. Cl. CPC A61M 11/007; G16H 20/13; A61M 2205/52; A61M 2205/3584; A61M 2205/8206

(57) ABSTRACT

Techniques for dosage delivery based on digital dosage of an aqueous ingestible solution are disclosed. Oral spray delivery of an ingestible substance is provided to an individual, where the ingestible substance is in an aqueous solution. The ingestible substance can be cannabidiol; or can be a pharmaceutical, antibiotic, vitamin, supplement, medicinal, extract, or legal psychoactive substance; a legal tetrahydrocannabinol; an essential oil; a mineral; melatonin; or caffeine. A spray plunger depression by the individual is detected to facilitate delivery of the ingestible substance. A digital signal is activated based on the detecting the spray plunger depression. The digital signal is transmitted to a device that tracks spray delivery of the ingestible substance to the individual. The device includes a software application that tracks the spray delivery of the ingestible substance. The software application provides a reminder to the individual for a next spray delivery.

### RELATED APPLICATIONS

This application claims the benefit of U.S. provisional patent application “Digital Dosage of Aqueous Medicinal Solutions” Ser. No. 62/991,119, filed Mar. 18, 2020.

The foregoing application is hereby incorporated by reference in its entirety.

### FIELD OF ART

This application relates generally to dosage delivery and more particularly to digital dosage of aqueous medicinal solutions.

### BACKGROUND

For centuries, elixirs, potions, and wonder cures have been hawked as remedies for a wide range of health conditions, both real and imagined. These items were promised to cure recurring complaints; to stop, cure, or prevent transmissible diseases; to extend life; or to rekindle the affections of a disinterested partner; among many other claims. For the last few centuries, “patent medicines” have joined the fray, each promising to perform better than all other offerings and to cure any and all complaints. These so-called remedies or nostrums often contained ingredients that were questionable at best. These ingredients were at the least ineffective in solving or curing a listed ailment, and at worst, downright lethal. The patent medicines were aggressively marketed to potential customers without any scientific evidence of efficacy, paid or planted endorsements notwithstanding. As a result, sellers of such products were fittingly labeled as “snake oil” salesmen. This epithet persists to this day and is still applied when unfounded, unproven, or outlandish claims regarding a product are bandied about. More recently, grocery, drug, and health store aisles and online websites are filled with vitamins, supplements, probiotics, and other over-the-counter items that infer improved health and wellbeing if taken regularly. Some of these items have been shown to have beneficial outcomes when used regularly and have actually been tested by government agencies that oversee such products. Other items are yet unproven, but enjoy a loyal and enthusiastic following. Whether the “benefits” extolled by the enthusiastic followers are real or imagined have yet to be determined.

From time to time, individuals experience complaints such as aches and pains; sniffles from a cold, influenza, or allergies; injuries of various severities; illnesses; and diseases. While some of these experiences are minor and can perhaps be ignored or can be treated with widely available over-the-counter medications such as pain relievers or decongestants, other complaints are more serious and require immediate intervention by a medical practitioner or health professional. These more serious complaints include injuries or diseases and may require further treatment including surgery or therapy, and may also require stronger, prescription medications. Notwithstanding injury or disease, individuals are also interested in improving and maintaining their general health. Proper diet and regular exercise have been demonstrated to be and are generally regarded as positive factors that contribute to good health. Further, many people want to more actively participate in maintaining and improving their health. As a result, people continue to seek vitamins and other dietary supplements, herbal remedies, “probiotics”, and more. Taking various supplements and remedies with the aim of predicting or predetermining one's future health and wellbeing is not new. Shakespeare noted use of, “eye of newt and toe of frog”, and other unsavory ingredients, in the witches' bubbling cauldron to predict the future. While some supplements and remedies have been shown to alleviate or control symptoms such as pain or anxiety, to control episodes such as seizures, or to generally have a positive effect on health, other items have not been evaluated scientifically.

### SUMMARY

Ingestible substances of many kinds are taken by individuals with the objectives of maintaining or improving health, controlling or regulating medical conditions, supplementing their diets, obtaining mild stimulants such as caffeine, and so on. Many of these ingestible substances are available over the counter at drug or grocery stores, health or natural food emporiums, and the like. Other medicinal substances are taken under a doctor's supervision to control medical conditions such as allergies and asthma, to counteract side effects of treatments such as chemotherapy or radiation therapy, to relieve pain after surgery, etc. These substances, whether medicinal substances by prescription, or over-the-counter herbal or supplemental ones, are available in a variety of forms. Substance forms include powders, tablets, capsules, liquids, food, and so on. While these substance forms are commonly available, ingestible substances that can be included in an aqueous solution can greatly enhance the experience of the individual ingesting the substance. The aqueous solution enables the substance to be delivered as a spray to the individual. While providing spray delivery of the ingestible substances can be a convenient delivery technique, the individual must keep track of dosage recommendations, frequency of the doses, timing of the doses, and the like. Further, the individual must remember to monitor the amount of ingestible substance remaining so that she or he can reorder more of the ingestible substance, refill a prescription, etc.

Disclosed techniques for dosage delivery are based on a digital dosage of an aqueous ingestible solution. Oral spray delivery of an ingestible substance is provided to an individual, where the ingestible substance is suspended in an aqueous solution. The medicinal substance can include a pharmaceutical, an antibiotic, a vitamin, a supplement, a medicinal, an extract, a legal psychoactive substance, a legal tetrahydrocannabinol, an essential oil, a mineral, melatonin, caffeine, and so on. A spray plunger depression by the individual is detected to facilitate delivery of the ingestible substance. The plunger depression can include depressing a button or lever, squeezing a bulb, energizing an actuator with an electrical button, and the like. A digital signal is activated based on the detecting the spray plunger depression. The digital signal can be based on a number, a code, a message, a flag, etc. The digital signal is transmitted to a device that tracks the spray delivery of the ingestible substance to the individual. The device can include a server, a computer, a portable device, a handheld device, etc. The device can include a smartphone, tablet, or PDA; a laptop or desktop computer; a cloud-based service; and so on. The transmitting the digital signal can be based on Wi-Fi™, Bluetooth™, Zigbee, etc. The tracking the spray delivery of the ingestible substance can be accomplished using a software application on the mobile device. The software application can provide a reminder to the individual. These reminders can include a calendar alert, popup reminder, or alarm. Further, the software application can record a circumstance experienced by the individual such as sleep tracking, heart rate tracking, or blood oxygen level; can alert the individual to the need to reorder or obtain further ingestible substance; and so on.

A processor-implemented method for dosage delivery is disclosed comprising: providing oral spray delivery of an ingestible substance to an individual, wherein the ingestible substance is in an aqueous solution; detecting a spray plunger depression by the individual to facilitate delivery of the ingestible substance; activating a digital signal based on the detecting a spray plunger depression; and transmitting the digital signal to a device that tracks the spray delivery of the ingestible substance to the individual.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for digital dosage of aqueous ingestible solutions.

FIG. 2 illustrates a spray delivery device in a transport configuration.

FIG. 3 shows a cutaway of a spray delivery device in a usage configuration.

FIG. 4 illustrates spray delivery device components.

FIG. 5 shows a detachable cartridge including a reservoir.

FIG. 6 is a block diagram for a spray apparatus.

FIG. 7 is a block diagram for spray device and mobile device interactions.

FIG. 8 is a system diagram for dosage delivery of an aqueous ingestible solution.

### DETAILED DESCRIPTION

Techniques for dosage delivery based on a digital dosage of an aqueous medical solution are disclosed. Individuals routinely use ingestible solution delivery devices to take prescription medicines such as steroids in order to control a variety of medical conditions such as asthma, or to take commonly available over-the-counter (OTC) products such as allergy relief products. More recently, individuals are using products based on cannabidiol (CBD) substances to try to relieve pain, and to reduce anxiety and depression, among other claimed benefits. Dosage delivery devices have also been used to deliver tetrahydrocannabinol (THC) substances for pain relief due to glaucoma, to provide relief from side effects such as nausea and appetite loss associated with chemotherapy, to reduce anxiety from post-traumatic stress, and so on. The dosage delivery devices may include a counter or some other indicator to track a number of remaining doses, but such techniques provide no indication regarding such important information as a time of a previous dose delivery, a reminder that a dose is due, an amount of remaining ingestible solution, and so on. A digital dosage of aqueous solution technique is described for which a dosage delivery device communicates with a software application on a mobile device associated with the individual. The software application or app can be used to track the spray delivery of the ingestible substance and to provide reminders to the individual for a next spray delivery. The app can further determine the contents of a removable cartridge that can be coupled to the spray delivery device by scanning a barcode, a QR code, or some other identifier.

Digital dosage of an aqueous ingestible solution enables dosage delivery to an individual. Spray delivery of an ingestible substance is provided to an individual orally, where the ingestible substance is suspended in an aqueous solution. A spray plunger depression by the individual is detected to facilitate the delivery of the ingestible substance. A digital signal is activated based on the detecting the spray plunger depression. The digital signal is transmitted to a mobile device that tracks the spray delivery of the ingestible substance to the individual. The mobile device includes a software application that tracks the spray delivery of the ingestible substance. The software application provides a reminder to the individual for a next spray delivery.

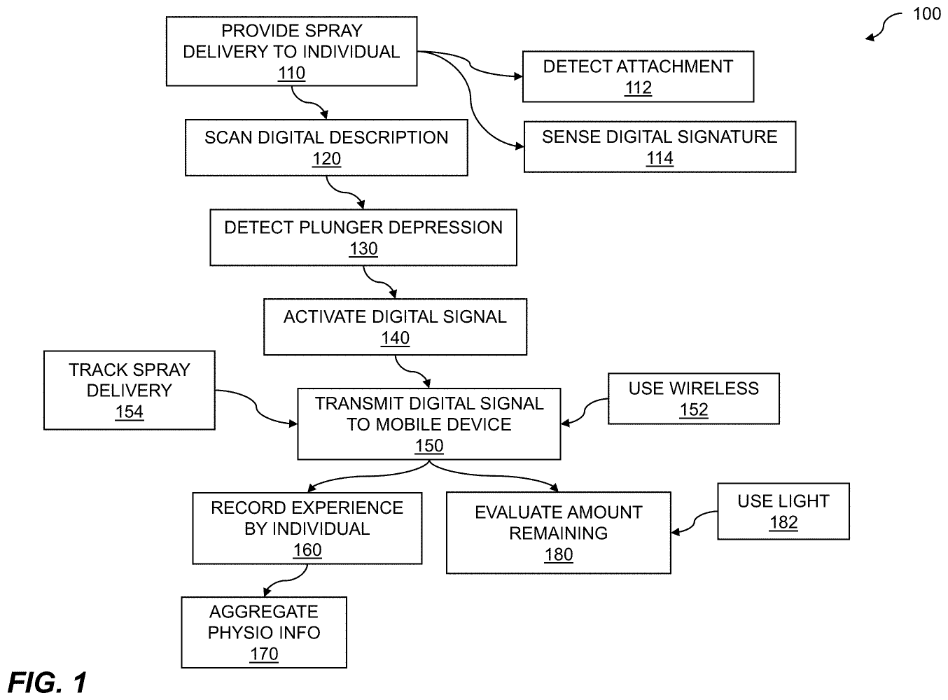

FIG. 1 is a flow diagram for a digital dosage of an aqueous ingestible solution. An aqueous ingestible solution can include a prescription substance, an over-the-counter substance, a recreational substance, and so on. The ingestible solution can be held within a reservoir and provided to an individual when the individual depresses a plunger associated with the spray delivery device. A time associated with a depression of the plunger by the individual, a duration since a previous plunger depression, ingestible substance dosage information, and so on, can be tracked by an application on a mobile device associated with the individual. The application can determine the various qualities of the ingestible substance, a quantity of ingestible substance and an amount remaining, etc. In addition to tracking plunger depressions, dosage amounts, dosage timing, and the like, the spray delivery device can record an experience associated with the ingestible substance. The recording the experience can include the tracking of sleep, heart rate, blood oxygen levels, and so on. The application can order additional ingestible substance when needed.

The flow 100 includes providing spray delivery of an ingestible substance to an individual orally 110 wherein the ingestible substance is in an aqueous solution. The ingestible substance can be suspended in the aqueous solution, dissolved in the aqueous solution, and so on. Discussed throughout, the ingestible substance can include a prescription medication (Rx), an over-the-counter (OTC) product, a recreational product, a caffeine product, a cannabidiol (CBD) product, a cannabigerol (CBG) product, a cannabinol (CBN) product, other cannabinoid extract products, an amino acid product, a nootropics product, a vitamin product, a ginseng product, a lavender product, a melatonin product, a pre-workout energy supplement product, a tea extract product, a natural fruit extract product, an essential oil product, a legal psychoactive substance product, a legal tetrahydrocannabinol (THC) product such as delta 8 THC, and so on. In embodiments, the ingestible substance can include a cannabidiol (CBD) substance. Many claims have been made regarding the salubrious qualities associated with CBD, including its use for treating seizure disorders such as epilepsy. Other claims propose using CBD for pain including chronic pain, anxiety, muscle disorders, Parkinson's disease, Crohn's disease, and so on. In other embodiments, the ingestible substance comprises a pharmaceutical, an antibiotic, a vitamin, a supplement, a medicinal, an extract, a legal psychoactive substance, a legal tetrahydrocannabinol, an extract, an essential oil, a mineral, melatonin, or caffeine. The ingestible substance can include legal tetrahydrocannabinol (THC) substances, which can be prescribed for treatment of chronic pain, the side effects of chemotherapy, anxiety, post-traumatic stress disorder, (PTSD), etc.

The flow 100 can include detection, by the spray delivery device, of attachment 112 of the detachable cartridge. The detection of the attachment can be accomplished using an electrical technique such as making or breaking a circuit, an optical technique such as blocking a light beam, and the like. The flow 100 further includes sensing a digital signature 114 from the detachable cartridge, where the digital signature can be associated with contents of the detachable cartridge. The digital signature can include a code such as a barcode, a number, etc. In embodiments, the digital description can include a quick response (QR) code. The digital signature can be used to convey a variety of information associated with the ingestible substance. In embodiments, the digital signature can identify a type of ingestible substance, a concentration of ingestible substance, or a metered dosage for an ingestible substance. The digital signature can further identify a manufacturer, a manufacture date, an expiration date, dosage recommendations, etc.

The flow 100 includes scanning by a mobile device of a digital description 120 of the ingestible substance. Discussed throughout, a mobile device can be associated with the spray delivery. The mobile device can execute an application associated spray delivery. The digital description of the ingestible substance can be scanned using a camera coupled to the mobile device. The scanning can include reading the digital signature described above. The scanning can include accessing a database, website, etc., associated with the QR code. The flow 100 includes detecting a spray plunger depression 130 by the individual to facilitate the delivery of the ingestible substance. The depression of the spray plunger can include pushing a button or lever, squeezing a bulb, pushing an electrical button associated with an actuator, touching a capacitive switch, and the like. The depressing the spray plunger can open a valve which can provide the ingestible substance to the individual. The flow 100 includes activating a digital signal 140 based on the detecting the spray plunger depression. The activating the digital signal can include activating a transmitter. The digital signal can include a sequence of discrete values. The digital signal can include a modulated signal based on various modulation techniques including digital signal modulation techniques. In embodiments, the digital signal modulation techniques can include phase-shift keying (PSK), frequency-shift keying (FSK), amplitude-shift keying (ASK), quadrature amplitude modulation (QAM), continuous phase modulation (CPM), and the like.

The flow 100 includes transmitting the digital signal to a device or mobile device 150. The device tracks the spray delivery 154 of the ingestible substance to the individual. The device can include a personal computer, a laptop computer, a smartphone, a tablet, a personal digital assistant (PDA), a smart watch, a purpose-built mobile device for dose tracking, and so on. The transmitting the digital signal can be accomplished using a variety of communications techniques and technologies. In embodiments, the digital signal that is transmitted can be based on wireless 152 communication technologies. Various wireless communication technologies such as technologies associated with computer communications, telephonic communications, automatic communications, etc. can be used. In embodiments, the wireless technology includes Bluetooth™. Other wireless communication techniques can be based on Wi-Fi™, Zigbee™, EnOcean™, near-field communication (NFC), etc. The digital signal can be sent through a wireless router directly to a cloud-based computing device, which can in turn be accessed by an application (app) residing on the individual's smart phone.

Returning to the device or mobile device, in embodiments, the mobile device can include a software application that tracks the spray delivery of the ingestible substance. The software application can include opensource software, an app downloaded from the Apple™ App Store™, the Google™ Play Store™, and the like. The software application or app can perform various operations and can provide information to the individual. In embodiments, the software application can provide a reminder to the individual for a next spray delivery. The reminder can include a smart device reminder, a calendar reminder, a text or SMS reminder, an email reminder, etc. The reminder can be based on various criteria, such as time, thresholds, dosage recommendations, and the like. In embodiments, the reminder can be based on time lapse since a previous spray delivery. The time lapse can include a number of hours, days, and the like. The reminder can be based on a number of times per day that a dose of an ingestible substance should be taken. In other embodiments, the reminder can be based on a condition described by the individual. In a usage example, the individual can describe a feeling of anxiety. The reminder can be based on a dosage recommendation to address the reported anxiety. In further embodiments, the reminder can be based on a therapeutic recommendation by a practitioner. A practitioner can prescribe a given number of doses of the ingestible substance to be taken a number of times per day. In other embodiments, the reminder can be based on an amount of a previous spray delivery. The reminder based on an amount of a previous spray can be used to address an underdose by the individual, a missed dose, an attempt to administer dosages more frequently than prescribed, etc.

The flow 100 further includes recording the experience, by the individual 160, where the experience is associated with the ingestible substance. The individual can report an experience such as the ingestible substance was easy to use, had certain side effects, and so on. The reported experience can include physiological data that reflects usage of the ingestible substance. The reported experience can include data collected by the spray delivery device, data reported by the individual, and so on. In embodiments, the experience can include sleep tracking. The individual can experience enhanced sleep, insomnia, and so on. Other physiological data can be collected. In embodiments, the experience can include heart rate tracking during exercise. The tracking of heart rate during exercise can be useful since the ingestible substance can include a stimulant, a depressant, etc. In other embodiments, the experience can include tracking blood oxygen levels, EKG findings, heart arrhythmia, or blood pressure. The flow 100 further includes aggregating physiological information 170 from a wearable device on the individual with dosage information on the ingestible substance. The aggregating the physiological information and the dosage information on the ingestible substance can be stored in the medical records for the individual, in a database such as a research database, in a cloud database, in a crowdsourced database, and so on. Other embodiments include crowd sourcing information on experiences of a plurality of people based on the ingestible substance.

The flow 100 further includes evaluating an amount of ingestible substance remaining 180 in a reservoir. The evaluating the amount of ingestible substance remaining can be based on techniques that include a remaining weight of the ingestible substance. The evaluating can be based on electrical techniques that can use electrical contacts. In embodiments, the evaluating the amount of substance remaining in the reservoir can be accomplished using a light 182 internal to a spray delivery device. The evaluating the amount of the ingestible substance remaining the reservoir can be used to determine that the individual is complying with usage guidelines associated with the ingestible substance. In embodiments, the software application can order additional ingestible substance based on usage. The ordering additional ingestible substance can include a refill, a regular order such as a monthly order or a quarterly order, etc. In other embodiments, the software application can provide a reminder when a reservoir of the ingestible substance is low. The reminder can be associated with an over-the-counter or other ingestible substance. Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

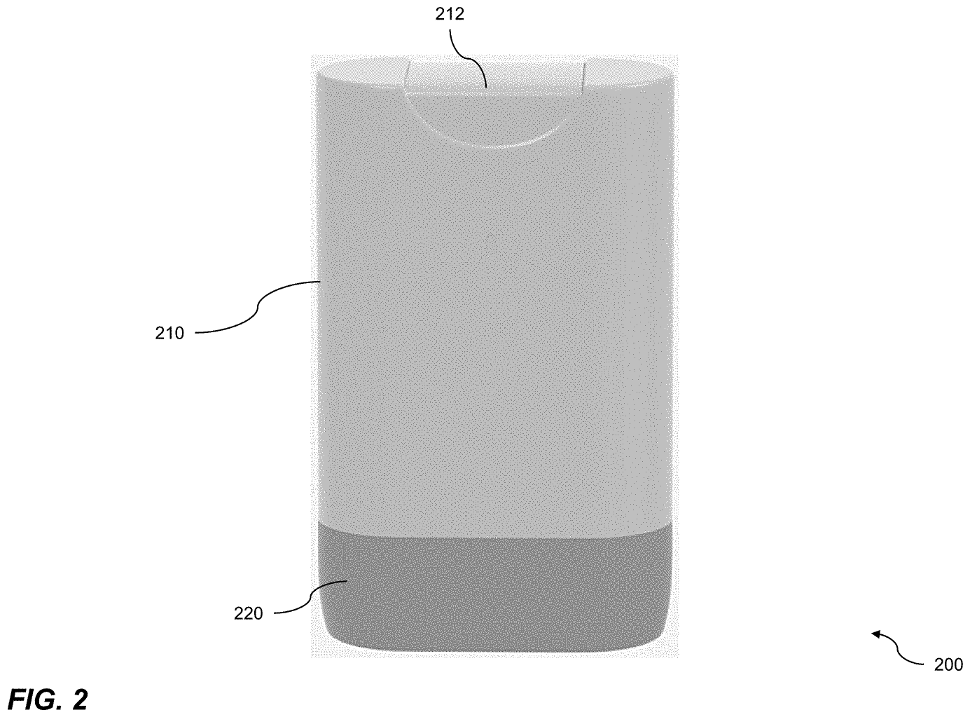

FIG. 2 illustrates a spray delivery device in a transport configuration. A spray delivery device, such as a portable spray delivery device, can be configured in a transport configuration. The transport configuration can include a compact or closed configuration, in which the spray delivery device is protected from dust and dirt, external contaminants, moisture, and so on. The transport configuration can include a technique for preventing the spray delivery device from being accidently discharged. The transport configuration enables safe transportation of the spray delivery device. The spray delivery device enables dosage delivery based on a digital dosage of an aqueous ingestible solution. Spray delivery of an ingestible substance is provided orally to an individual, where the ingestible substance is suspended in an aqueous solution. A spray plunger depression by the individual is detected to facilitate the delivery of the ingestible substance. A digital signal is activated based on the detecting the spray plunger depression, and the digital signal is transmitted to a mobile device that tracks the spray delivery of the ingestible substance to the individual. The ingestible substance can include a cannabidiol substance; a pharmaceutical, antibiotic, vitamin, supplement, or caffeine substance; etc.

The spray delivery device 210 can be configured in a transport configuration 200. Discussed above, the transport configuration can include a compact, closed, sealed, locked, and so on, configuration. The transport configuration can help keep the spray delivery device clean, protected from weather, safe from unauthorized use, and the like. The spray delivery device 200 can include a button 212. The button can be locked, blocked, or otherwise protected from depression while the spray delivery device is in the transport configuration. The spray delivery device can include a detachable cartridge 220. Discussed throughout, the detachable cartridge can include a reservoir which can contain the ingestible substance. The detachable cartridge can be protected while the spray delivery device is in a transport configuration. In embodiments, the transport configuration can use a locking technique to prevent the detachable cartridge from being detached from the spray delivery device. The spray delivery device can be reconfigured from transport configuration by removing a cover, repositioning one or more components of the spray delivery device, etc., and reattaching the cover in a different position. The spray delivery device can be reconfigured from transport configuration by a software application, discussed later.

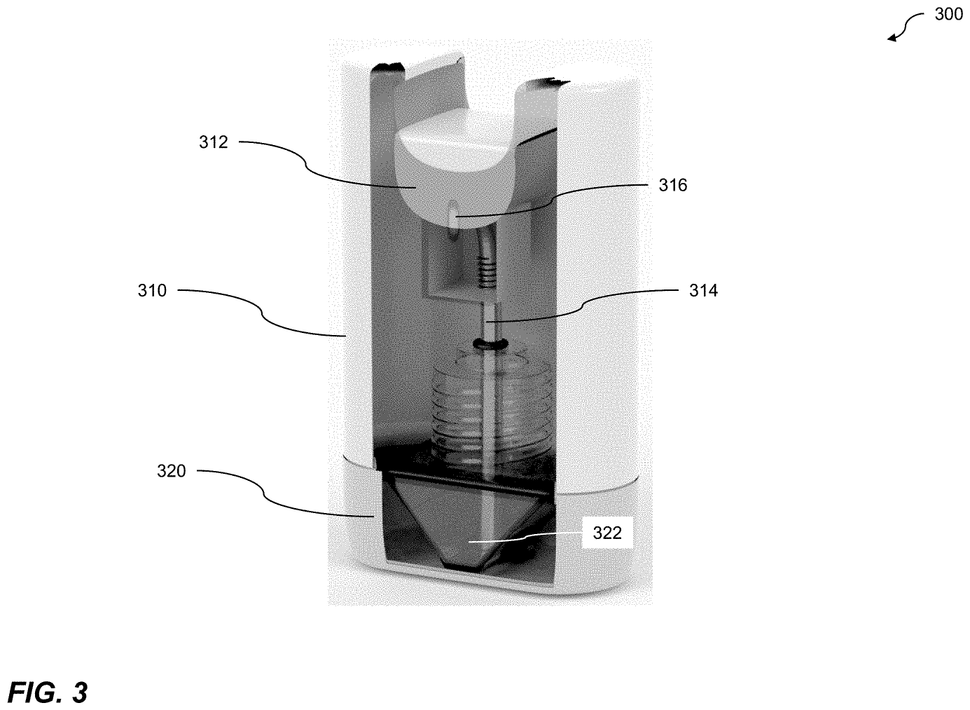

FIG. 3 shows a cutaway of a spray delivery device in a usage configuration 300. The spray delivery device can be configured for usage by sliding the device open, by removing a protective cover, and so on. The spray delivery device in a usage configuration enables dosage delivery of a digital dosage of an aqueous ingestible solution. Spray delivery of an ingestible substance is provided to an individual orally. A spray plunger depression by the individual is detected, and a digital signal is activated. The digital signal is transmitted to a mobile device that tracks the spray delivery. The spray delivery device 310 is shown in usage configuration 300. A button 312 is coupled to a plunger 314. The button is depressed by the individual to release an ingestible substance via a distribution port 316. The individual using the spray delivery device can place the spray delivery device adjacent to their mouth prior to depressing the button 312. As the individual depresses the button, the individual can inhale the ingestible substance that is released through the distribution port. A detachable cartridge 320 can be coupled to the spray delivery device prior to usage. The detachable cartridge can be coupled to the spray delivery device during transport, as discussed previously. The detachable cartridge can include a reservoir which has a conical base 322, where the conical base can enhance collection of the ingestible substance from the reservoir.

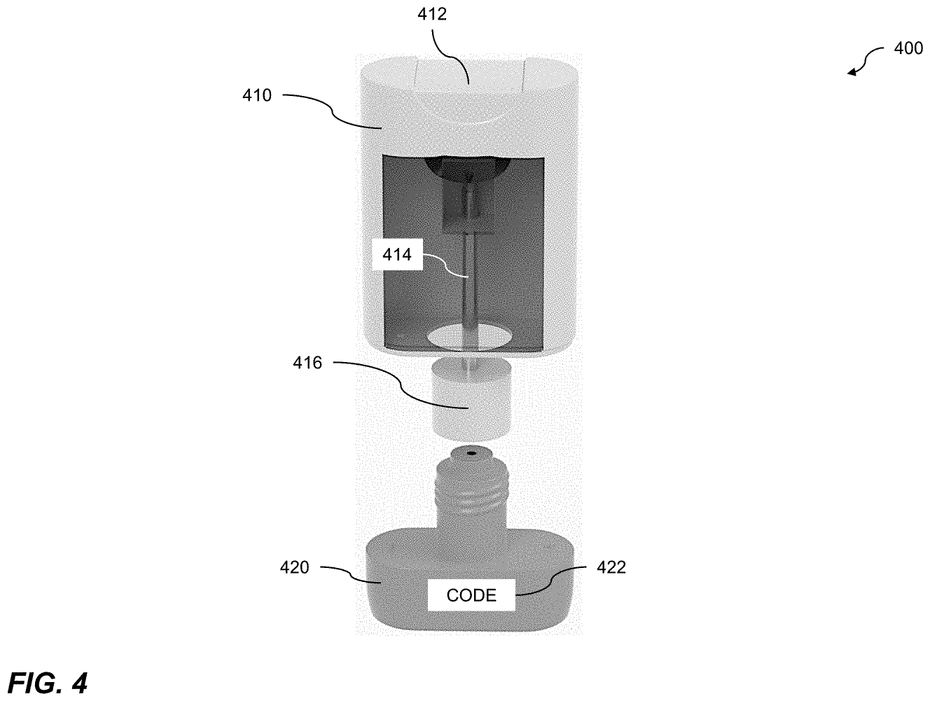

FIG. 4 illustrates spray delivery device components. A spray device 410 can be used to deliver, to an individual, various substances within solutions. The delivery of the spray can be based on a dosage including a digital dosage of an aqueous ingestible solution. Spray delivery of an ingestible substance suspended in an aqueous solution is provided to an individual. A spray plunger depression by the individual is detected to facilitate the delivery of the ingestible substance. A digital signal is activated based on the detecting the spray plunger depression, and the digital signal is transmitted to a mobile device that tracks the spray delivery.

A cutaway view 400 of components of a spray delivery device is shown. The spray delivery device includes a button 412 coupled to a plunger 414. While a mechanical button is shown, the button could be substituted with a bulb, an actuator controlled by an electrical button, a lever, and so on. The plunger 414 is coupled to a coupling or mount 416. The coupling can be used to receive a corresponding adapter, coupling, or mount associated with a detachable cartridge 420. The detachable cartridge can include a reservoir in which an ingestible substance can be included. Embodiments include detection, by the spray delivery device, of attachment of the detachable cartridge. The detection of attachment of the cartridge can be accomplished using electrical techniques, optical techniques, and so on. The detection of attachment of the cartridge can also be used to determine the contents of the detachable cartridge. Further embodiments include sensing a digital signature from the detachable cartridge where the digital signature is associated with contents of the detachable cartridge. The contents of the detachable cartridge can include a cannabidiol substance, or a pharmaceutical, antibiotic, vitamin, supplement, or caffeine substance, to name just a few possible substances. In embodiments, the digital signature can identify a type of ingestible substance, a concentration of ingestible substance, or a metered dosage for an ingestible substance. The sensing the digital signature can include a scanning code 422 such as a barcode, a quick response (QR) code, and the like.

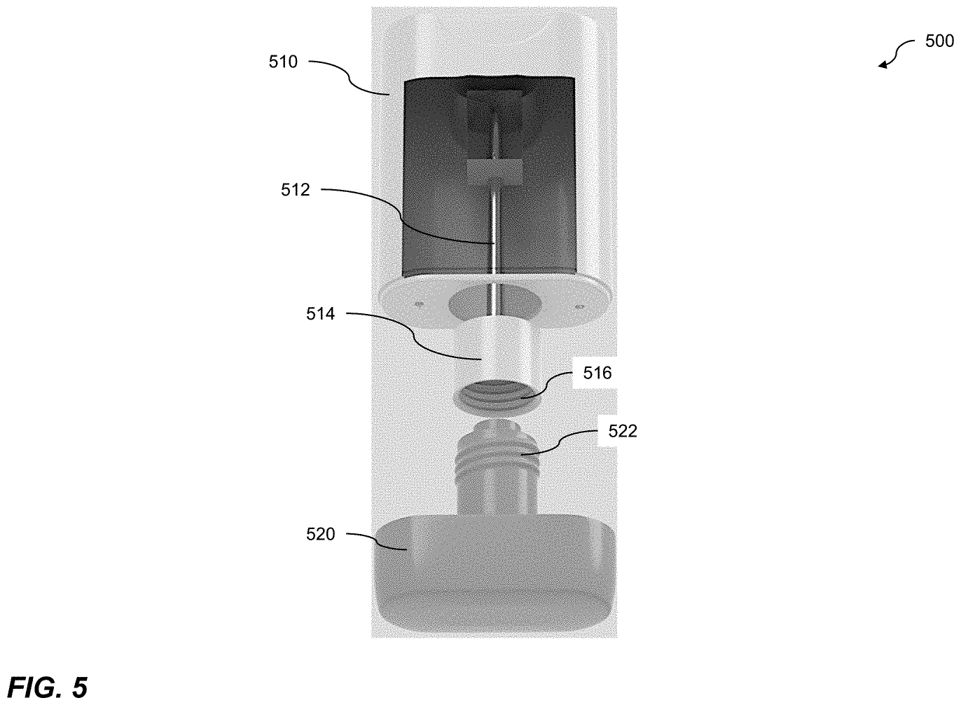

FIG. 5 shows a detachable cartridge including a reservoir. A detachable cartridge, including the reservoir, can contain an ingestible substance. The detachable cartridge provides the ingestible substance to enable dosage delivery to an individual. Spray delivery of an ingestible substance is provided to an individual orally, where the ingestible substance is suspended in an aqueous solution. A spray plunger depression by the individual, to facilitate the delivery of the ingestible substance, is detected. A digital signal is activated based on the detecting the spray plunger depression. The digital signal is transmitted to a mobile device that tracks the spray delivery of the ingestible substance to the individual. The ingestible substance can include a cannabidiol substance.

A spray delivery device 510 to which a detachable cartridge can be coupled is shown 500. Discussed throughout, the spray delivery device can include a plunger 512. While a plunger comprising a button coupled to a rod is shown, the plunger can include a bulb, an actuator operated by an electrical button, and so on. The plunger is depressed by an individual to receive a delivery of a spray that includes an ingestible substance. The plunger is coupled to a coupling 514. The coupling can be based on a locking mount technique, on a bayonet mount technique, and the like. In embodiments, the coupling is based on a threaded mount 516 technique. A detachable cartridge 520 can be coupled to the coupling 514. The detachable cartridge can include a reservoir that contains the ingestible substance. The detachable cartridge can include a corresponding coupling that mates with the coupling of the spray delivery device. In embodiments the corresponding coupling of the detachable cartridge can be based on a threaded mount technique 522.

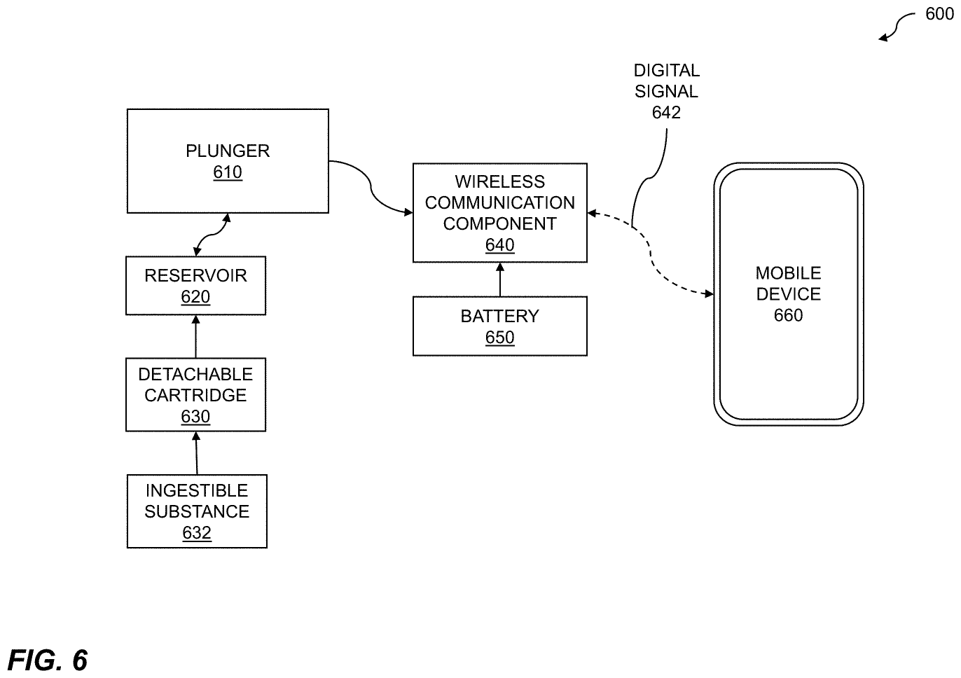

FIG. 6 is a block diagram for a spray apparatus. The spray apparatus enables spray delivery based on a digital dosage of an aqueous ingestible solution. The spray apparatus can be used for treatment of various medical conditions, for self-treatment, for recreation, and so on. Spray delivery of an ingestible substance is provided to an individual orally. The ingestible substance is suspended in an aqueous solution. A spray plunger depression by the individual is detected to facilitate the delivery of the ingestible substance. A digital signal is activated based on the detecting the spray plunger depression. The digital signal is transmitted to a mobile device that tracks the spray delivery of the ingestible substance to the individual. The mobile device includes a software application that tracks the spray delivery of the ingestible substance.

A block diagram for a spray apparatus 600 is shown. The spray apparatus 600 includes a plunger 610. The plunger can include a button, a lever, a bulb, an electrically controlled actuator, and so on. In embodiments, depressing the plunger can provide spray delivery of an ingestible substance to an individual orally. The ingestible substance can include a cannabidiol substance. The ingestible substance can include prescribed substances, over-the-counter substances, recreational substances, and so on. The ingestible substance is suspended in an aqueous solution. In other embodiments, the ingestible substance can include a pharmaceutical, an antibiotic, a vitamin, a supplement, a caffeine substance, etc. The spray apparatus 600 includes a reservoir 620. The reservoir can include the ingestible substance in an aqueous solution. The reservoir can have a curved bottom interior surface, where the curved bottom interior surface can enhance collection of the medicinal substance to a low spot of the reservoir for better accessibility to a plunger tube. The reservoir can have a flat bottom interior surface, which could help maximize the amount of aqueous solution containable in the reservoir. The reservoir can be monitored by the spray device. Embodiments can include evaluating an amount of substance remaining in a reservoir. Evaluating the amount of substance remaining in the reservoir can be based on weight, volume, and so on. In embodiments, evaluating the amount of substance remaining in the reservoir can be accomplished using a light internal to a spray delivery device.

The spray apparatus 600 includes a detachable cartridge 630. The detachable cartridge can be coupled to the spray device using threads, a bayoneted mount, a locking technique, and so on. Embodiments can include detection, by the spray delivery device, of the attachment of the detachable cartridge. The detection can be based on an electrical connection, optical detection, and so on. The detachable cartridge can contain the ingestible substance 632. In embodiments, the detachable cartridge can include the reservoir. Since detachable cartridges can include a variety of ingestible substances, a particular ingestible substance within a given detachable cartridge can be identified. Further embodiments can include sensing a digital signature from the detachable cartridge wherein the digital signature is associated with contents of the detachable cartridge. The digital signature can include an amount of the ingestible substance, a source, a manufacturer, etc. In embodiments, the digital signature can identify a type of ingestible substance, a concentration of ingestible substance, or a metered dosage for an ingestible substance.

The spray apparatus 600 includes a wireless communication component 640, coupled to the plunger, that communicates a digital signal based on the plunger being depressed. The wireless communication component can support variety of digital and analog communication technologies. In embodiments, the wireless technology includes Bluetooth™. The wireless technology can also include Wi-Fi™, Zigbee™, NRC, IR, etc. The digital signal 642 can be transmitted to a mobile device (discussed below) that tracks the spray delivery of the ingestible substance to the individual. The spray apparatus 600 includes a battery 650, coupled to the wireless communication component that powers the wireless communication component. The battery can include a replaceable such as an alkaline battery, a rechargeable battery such as a lithium-ion battery or a nickel-metal-hydride battery, etc. In embodiments, recharging the rechargeable battery can be accomplished using a wireless charger such as an induction charger. The induction charger can be based on the Qi™ charging standard or some other standard. In other embodiments, recharging the battery can be accomplished using a USB connection. The digital signal that is transmitted can be received by a mobile device 660. The mobile device can include a laptop computer, a smartphone, a tablet, a PDA, a monitoring device, and so on. A software application or app can be executed on the mobile device to present information relating to the spray device, the ingestible substance, and so on, to an individual using both the spray device and the mobile device. In other embodiments, spray delivery is accomplished by a spray delivery device comprising a plunger, an aqueous solution reservoir, a battery, a wireless communication component, and a detachable cartridge comprising the reservoir.

In embodiments, an apparatus for dosage delivery comprising: a plunger, wherein depressing the plunger provides oral spray delivery of an ingestible substance to an individual, wherein the ingestible substance is in an aqueous solution; a reservoir, coupled to the plunger, containing the ingestible substance in an aqueous solution; a wireless communication component, coupled to the plunger, that communicates a digital signal based on the plunger being depressed, wherein the digital signal is transmitted to a device that tracks the spray delivery of an ingestible substance to the individual; a battery, coupled to the wireless communication component that powers the wireless communication component; and a detachable cartridge comprising the reservoir.

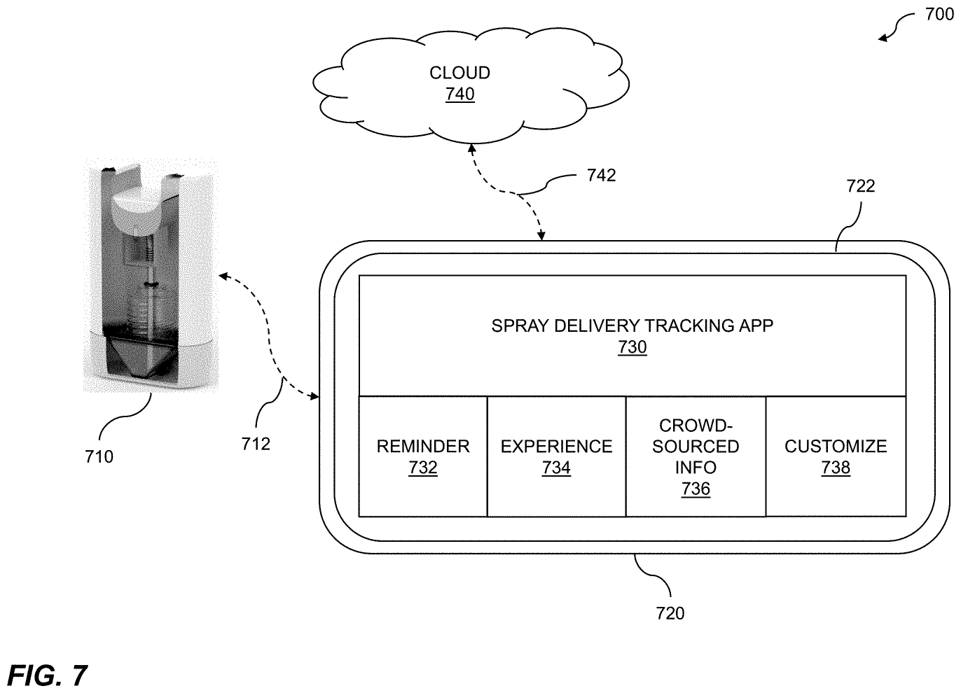

FIG. 7 is a block diagram for spray device and mobile device interactions. A spray device, such as a spray device that includes a digital dosage of aqueous ingestible solution, enables dosage delivery of the aqueous ingestible solution. The spray device can interact with a mobile device, personal electronic device, and so on, to track usage, dosages, response by the individual, and so on. A spray delivery of an ingestible substance is provided to an individual orally, where the ingestible substance is suspended in an aqueous solution. A spray plunger depression by the individual is detected, which facilitates the delivery of the ingestible substance. A digital signal is activated based on the detecting the spray plunger depression. The digital signal is transmitted to a mobile device that tracks the spray delivery of the ingestible substance to the individual.

An example system for spray device and mobile device interactions is shown 700. The system can include a spray device 710. Discussed throughout, the spray device can provide a digital dosage of an aqueous ingestible solution. The ingestible substance can include a cannabidiol substance; a THC substance; a pharmaceutical, antibiotic, vitamin, supplement, or caffeine substance; and so on. The spray device can be activated by pressing a plunger or lever, squeezing a bulb, pressing an electronic button, etc. The spray device can transmit a digital signal 712. The digital signal can include a number, a tone, a code, and so on. The digital signal can be modulated based on various modulation techniques including digital signal modulation techniques. In embodiments, the digital signal modulation techniques can include phase-shift keying (PSK), frequency-shift keying (FSK), amplitude-shift keying (ASK), quadrature amplitude modulation (QAM), continuous phase modulation (CPM), and the like. In other embodiments, the digital signal that is transmitted can be based on wireless communication technologies including Wi-Fi™, Bluetooth™, Zigbee™, near-field communication (NFC), etc.

The spray device can communicate with a device, and particularly, a mobile device 720. The mobile device can include a laptop, a smartphone, a tablet, a personal digital assistant (PDA), a smart watch, and so on. A digital display 722 can be coupled to the mobile device. The digital display can be used to present rendered information related to use of the spray device to an individual. The rendering of the information related to the use of the spray device can be accomplished using software, where the software can include an application, an app, and the like. In embodiments, the mobile device can include a software application 730 that tracks the spray delivery of the ingestible substance. The software application can present a variety of information to the individual. In embodiments, the software application can provide a reminder 732 to the individual for a next spray delivery. The reminder can be based on a time, a date, a dosage, and so on. In a usage example, the reminder can be based on a usage recommendation such as taking the ingestible substance three times per day. In embodiments, the reminder can be based on time lapse since a previous spray delivery, such as taking the ingestible substance every four hours. The reminder can be based on symptoms or other information reported by the individual. In other embodiments, the reminder can be based on a condition described by the individual. In a usage example, the condition reported by the individual can include, “I have a cold today,” or “I am feeling anxious.” The reminder provided to the individual can be based on a treatment, a therapy, and so on. In further embodiments, the reminder is based on a therapeutic recommendation by a practitioner. The practitioner can include a doctor, a therapist, an advisor, etc. The reminder can be based on further usage, recommendations, parameters, and so on. In embodiments, the reminder can be based on an amount of a previous spray delivery. The amount of a previous spray delivery can be based on a recommended amount, a self-prescribed amount, and so on. The previous spray delivery can be based on an under-delivery of a dose or an over-delivery of a dose. The previous spray delivery can be used to avoid abuse of the ingestible substance.

The software application can be used to collect usage and other data associated with usage of the spray device. Further embodiments can include recording an experience 734, by the individual, where the experience is associated with the ingestible substance. The experience can include the efficacy of the ingestible substance to alleviate symptoms, reactions to the ingestible substance, etc. In embodiments, the experience can include sleep tracking. For example, sleep tracking can track whether the individual sleeps better, the same, or worse while using the ingestible substance, and it can be compared to the amount of consumption, the frequency of consumption, the timing of consumption, etc. The experience can include tracking other physiological parameters. In further embodiments, the experience can include tracking blood oxygen level, EKG, heart arrhythmia, blood pressure, and so on. In addition to physiological parameters, psychological parameters can also be monitored. The software can provide other information to the individual user of the spray device. In embodiments the software application can present crowdsourced information to the individual 736. The crowdsourced information can include group discussions, peer support, journal or popular articles, and the like. Further embodiments include crowdsourcing information on experiences of a plurality of people based on the ingestible substance. The mobile device 720 can interact with a cloud 740. The cloud can include the Internet, a private network provided to support usage of the spray device, and so on. The mobile device can communicate with the cloud using a wireless or other connection 742.

The software application can enable customized formulae 738 of one or more ingestible substances. Various experience 734 and crowdsourced information 736 can be included in the creation of a customized formula of one or more ingestible substances. For example, other individual users may have discovered that a particular vitamin combined with a particular essential oil can yield desirous results. The individual can use the software application to order that particular combination for personal use. Thus, embodiments provide customizing a formula of the ingestible substance by the individual. The customizing can also include controlling aspects of the spray device, such as reconfiguring the spray device from a transport mode to a use mode.

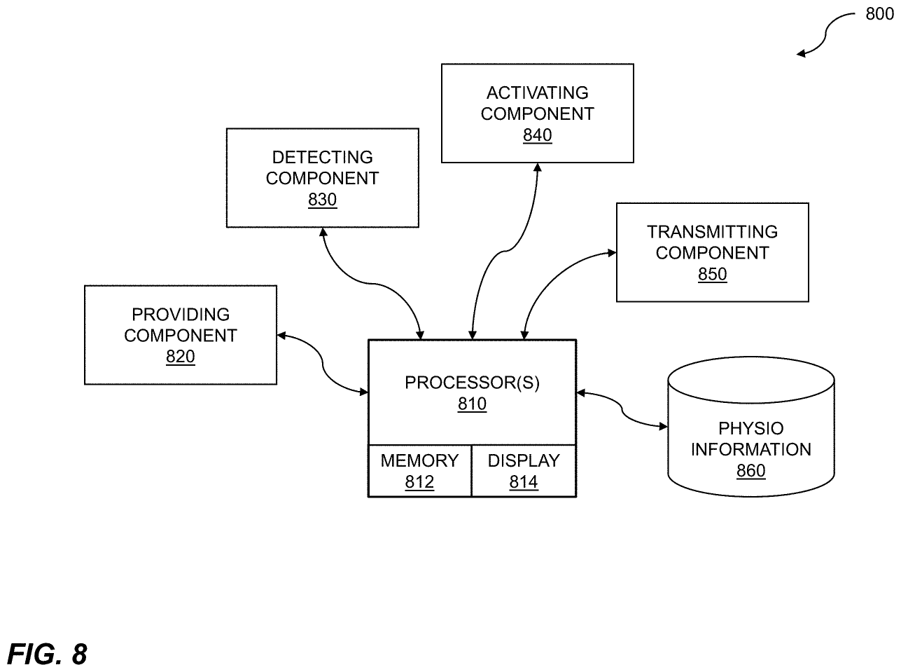

FIG. 8 is a system diagram for dosage delivery of an aqueous ingestible solution. The system 800 can include one or more processors 810 attached to a memory 812 which stores instructions. The system 800 can include a display 814 coupled to the one or more processors 810 for displaying data, videos, intermediate steps, instructions, short-form videos, and so on. In embodiments, one or more processors 810 are attached to the memory 812 where the one or more processors, when executing the instructions which are stored, are configured to: provide spray delivery of a ingestible substance to an individual orally wherein the ingestible substance is suspended in an aqueous solution; detect a spray plunger depression by the individual to facilitate the delivery of the ingestible substance; activate a digital signal based on the detecting the spray plunger depression; and transmit the digital signal to a mobile device that tracks the spray delivery of the ingestible substance to the individual.

The system 800 can include a providing component 820. The providing component 820 can provide a spray delivery of an ingestible substance. In embodiments, the ingestible substance can include a cannabidiol (CBC) substance. The CBD substance can be extracted from hemp or other related plant sources. The CBD substance can be used to treat a variety of syndromes such as childhood epilepsy. In other embodiments, the ingestible substance can include a pharmaceutical, antibiotic, vitamin, supplement, or caffeine substance. These substances can include prescription medications, over-the-counter (OTC) products, stimulants, and the like. In further embodiments, the ingestible substance can include tetrahydrocannabinol (THC). The THC can be used for medical purposes such as to relieve nausea and vomiting due to chemotherapy or movement disorders, pain from glaucoma, psychiatric symptoms, etc. The system 800 can include a detecting component 830. The detecting component 830 can detect a spray plunger depression by the individual. The spray plunger depression can include pushing a button or lever, squeezing a bulb, and the like. The detecting the depression can be based on closing or opening electrical contacts, transmitting or blocking light, etc.

The system 800 can include an activating component 840. The activating component 840 can activate a digital signal based on the detecting the spray plunger depression. The digital signal can include a number, a code, a pulse code modulated (PCM) signal, and so on. The system 800 can include a transmitting component 850. The transmitting component can transmit the activated digital signal from a dosage delivery system to an electronic device such as a smartphone, a tablet, a PDA, a laptop computer, and the like. In embodiments, the transmitting the digital signal can be accomplished using wireless technology. The wireless technology can include a wireless technology used to communicate between computers; personal electronic devices such as smartphones, tablets, or PDAs; and the like. The wireless technology can include Wi-Fi™, Bluetooth™, Zigbee™, Near-Field Communication (NFC), infrared (IR), etc. The system 800 can include physiological information 860. The physiological information can include body temperature, heart rate, heart rate variability, respiratory rate, blood oxygen levels, blood pressure, and so on. The physiological information can be stored in a library, a database, a distributed database, etc. The physiological information can be aggregated. Embodiments include aggregating physiological information from a wearable device on the individual with dosage information on the ingestible substance. The physiological information and the dosage information on the ingestible substance can be analyzed so that the dosage information can be adjusted based on a physiological response by the individual to the ingestible substance.

The system 800 can include a computer program product embodied in a non-transitory computer readable medium for dosage delivery, the computer program product comprising code which causes one or more processors to perform operations of: providing oral spray delivery of an ingestible substance to an individual, wherein the ingestible substance is in an aqueous solution; detecting a spray plunger depression by the individual to facilitate delivery of the ingestible substance; activating a digital signal based on the detecting a spray plunger depression; and transmitting the digital signal to a device that tracks the spray delivery of the ingestible substance to the individual.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”— may be implemented by computer program instructions, by special-purpose hardware-based computer systems, by combinations of special purpose hardware and computer instructions, by combinations of general-purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are limited to neither conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for dosage delivery comprising:
providing oral spray delivery of an ingestible substance to an individual, wherein the ingestible substance is in an aqueous solution;
detecting a spray plunger depression by the individual to facilitate delivery of the ingestible substance;
activating a digital signal based on the detecting a spray plunger depression; and
transmitting the digital signal to a device that tracks the spray delivery of the ingestible substance to the individual.

2. The method of claim 1 wherein the ingestible substance comprises a cannabidiol.

3. The method of claim 1 wherein the ingestible substance comprises a pharmaceutical, an antibiotic, a vitamin, a supplement, a medicinal, an extract, a legal psychoactive substance, a legal tetrahydrocannabinol, an essential oil, a mineral, melatonin, or caffeine.

4. The method of claim 1 wherein the device includes a software application that tracks the spray delivery of the ingestible substance.

5. The method of claim 4 wherein the software application provides a reminder to the individual for a next spray delivery.

6. The method of claim 5 wherein the reminder is based on time lapse since a previous spray delivery.

7. The method of claim 5 wherein the reminder is based on a condition described by the individual.

8. The method of claim 5 wherein the reminder is based on a therapeutic recommendation by a practitioner.

9. The method of claim 5 wherein the reminder is based on an amount of a previous spray delivery.

10. The method of claim 4 further comprising recording an experience, by the individual, wherein the experience is associated with the ingestible substance.

11-13. (canceled)

14. The method of claim 4 wherein the software application orders additional ingestible substance based on usage.

15. The method of claim 4 wherein the software application provides a reminder when a reservoir of the ingestible substance is low.

16. The method of claim 4 further comprising crowdsourcing information on experiences of a plurality of people based on the ingestible substance.

17. The method of claim 1 further comprising evaluating an amount of aqueous solution remaining in a reservoir.

18. The method of claim 17 wherein the evaluating the amount of aqueous solution remaining in the reservoir is accomplished using a light internal to a spray delivery device.

19. The method of claim 1 wherein spray delivery is accomplished by a spray delivery device comprising a plunger, an aqueous solution reservoir, a battery, a wireless communication component, and a detachable cartridge comprising the reservoir.

20-23. (canceled)

24. The method of claim 19 wherein the detachable cartridge includes the reservoir.

25. The method of claim 19 further comprising detecting, by the spray delivery device, attachment of the detachable cartridge.

26. The method of claim 25 further comprising sensing a digital signature from the detachable cartridge, wherein the digital signature is associated with contents of the detachable cartridge.

27. The method of claim 26 wherein the digital signature identifies a type of ingestible substance, a concentration of ingestible substance, or a metered dosage for ingestible substance.

28. The method of claim 1 wherein the device is a mobile device.

29. The method of claim 28 further comprising scanning, by the mobile device, a digital description of the ingestible substance.

30. (canceled)

31. The method of claim 1 further comprising aggregating physiological information from a wearable device on the individual with dosage information on the ingestible substance.

32. The method of claim 1 further comprising customizing a formula of the ingestible substance by the individual.

33. A computer program product embodied in a non-transitory computer readable medium for dosage delivery, the computer program product comprising code which causes one or more processors to perform operations of:
providing oral spray delivery of an ingestible substance to an individual, wherein the ingestible substance is in an aqueous solution;
detecting a spray plunger depression by the individual to facilitate delivery of the ingestible substance;
activating a digital signal based on the detecting a spray plunger depression; and
transmitting the digital signal to a device that tracks the spray delivery of the ingestible substance to the individual.

34. An apparatus for dosage delivery comprising:
a plunger, wherein depressing the plunger provides oral spray delivery of an ingestible substance to an individual, wherein the ingestible substance is in an aqueous solution;
a reservoir, coupled to the plunger, containing the ingestible substance in an aqueous solution;
a wireless communication component, coupled to the plunger, that communicates a digital signal based on the plunger being depressed, wherein the digital signal is transmitted to a device that tracks the spray delivery of an ingestible substance to the individual;
a battery, coupled to the wireless communication component that powers the wireless communication component; and
a detachable cartridge comprising the reservoir.
